(19) United States
(12) Patent Application Publication
(10) Pub. No.: US 2016/0317023 A1
(43) Pub. Date: Nov. 3, 2016

(54) SYSTEM FOR TESTING VISUAL FIELD OF A PATIENT AND THE CORRESPONDING METHOD

(71) Applicant: METROVISION, Perenchies (FR)

(72) Inventor: Jacques Charlier, Villeneuve d'Ascq (FR)

(21) Appl. No.: 14/698,980

(22) Filed: Apr. 29, 2015

(51) Int. Cl. A61B 3/024; A61B 3/113; A61B 3/14; A61B 3/00

(52) U.S. Cl. CPC A61B 3/024; A61B 3/0058; A61B 3/113; A61B 3/145

(57) ABSTRACT

Disclosed are systems and devices for testing the visual field of a patient. The components and functions may include means for selecting the features of successive visual stimuli, a display for presenting said successive visual stimuli to the patient; a video recorder for recording video of movements of at least part of the patent's head, and in particular movements of the patient's eyes, together with time codes, during the presentation of said visual stimuli, and a data recorder for recording, for each visual stimulus, the features and at least one time-code of the video recorder corresponding to the displayed visual stimulus. Various embodiments also provide methods for testing the visual field of a patient.

### BACKGROUND OF THE INVENTION

The present invention relates to a system and method for testing visual field of a patient. The invention is particularly suited to testing visual fields in children.

The testing methods of the visual field, usually referred to as methods of perimetry, and the corresponding systems, usually referred to as perimeters, are used for the detection and monitoring of many eye conditions and diseases: glaucoma, optic neuropathy, vascular disease of the retina, macular degeneration, retinitis pigmentosa, monitoring in the case of risk of drug intoxication, etc. These testing methods are usually performed under monocular conditions, i.e. one eye at a time.

The detection of visual field defects is crucial in the management of children with medical conditions such as retinopathy of prematurity, retinitis pigmentosa, glaucoma, cerebral visual impairment, cerebral tumor, raised intracranial pressure and Vigabatrin (Sabril) treatment. Unfortunately, most existing methods of perimetry are not suitable for children. Children have different needs and requirements from adults when it comes to perimetry, because of their reduced ability to learn tasks and to provide appropriate responses during conventional testing. Children are also less inclined to cooperate due to a lack of understanding of the test methods and their short attention span.

In adults, the reliability of most standard visual field testing methods is dependent upon the ability of the subject to learn the task asked of the during the test. The two main tasks are to fixate on a central target and to indicate by, for example, pressing a button when they see a light stimulus in their field of vision. In order to keep a continuous fixation on a central target when a stimulus is presented in their visual field, the subject needs conscious effort to inhibit their natural response to fixate on the stimulus, thereby preventing the loss of fixation on the central target. It is much more difficult for children, especially children below the age of five, to inhibit the natural saccadic reaction that is triggered by light stimuli in the visual field.

Moreover, it is also important, during a visual field test, to visualize the patient's eye throughout the test. Indeed, this allows the control of several factors that affect the results of the visual field test and their reliability. Such factors are for instance:
the size of the pupil and its fluctuations
abnormal eye movements such as nystagmus
the opening of the eye lids or presence of a ptosis
the position of the lens used to correct refractive errors
abnormal head position and head movements.

At present, the recording of these factors depends on the operator performing the test, and these factors are mainly lost at the end of the test. The interpretation of the test, which is usually carried out by another person after the test, can be challenging if the operator has not indicated precisely these factors during the test.

Moreover, for some difficult patients such as children or people with low cooperation, the operator does not have enough time to take care of the patient, control the tests and simultaneously mention the factors.

Today, there are mainly two kinds of systems for testing the visual field: manual systems and automated systems.

Manual systems, such as the Goldmann system, comprise a hemispherical dome illuminated uniformly on which an opto-mechanical device projects a light spot. The position, size and luminance of this light spot are controlled manually by the operator by means of a pantograph. The operator also monitors the immobility of the eye with a telescopic device and records the subject's responses using colored pencils.

However, manual systems are not fully satisfying as they require more qualified and experienced staff.

Automated systems comprise a computer that automatically performs the tests, controls the eye position, records the patient's responses, performs several analyses such as the comparison with normal values, and monitors changes over several successive tests. Such automated systems may comprise for example means (electrodes or a camera) placed on the patient's head to detect movements of patient's gaze or patient's head but not both. Another example of automated system is described in document EP 2 148 609 which discloses a system for testing visual fields in which the position of the stimulus on the display depends on the position of the patient's eye determined by an eye tracker.

However, automated systems do not allow performing tests on certain patients (e.g., uncooperative, tired, with large attention deficits). For these patients, automated systems are less reliable and are slower than manual systems.

### OBJECTS AND SUMMARY OF THE INVENTION

An object of the present invention is to solve the various above-listed technical problems. In particular, an object of the present invention is to propose a system and a method allowing an interpretation of the results after the test. Another object of the invention is to propose a system and a method allowing performing the test on uncooperative patients, in particular young children.

Thus, in one aspect, the invention provides a system for testing visual field of a patient, comprising:
means for selecting the features of successive visual stimuli,
a display for presenting said successive visual stimuli to the patient;
a video recorder for recording video of movements of at least part of patient's head, and in particular movements of patient's eyes, together with time codes, during the presentation of said visual stimuli, and
a data recorder for recording, for each visual stimulus, the features and at least one time-code of the video recorder corresponding to the displayed visual stimulus.

By means of the recording of the video images of movements of at least part of patient's head, and in particular movements of patient's eyes, in synchrony with the visual field test, the system according to the invention provides means for documenting and reviewing the events occurring during visual field tests. It thus gives the possibility to replay the visual field test afterwards, for instance by a person other than the operator. It is no longer necessary for the operator to precisely record the factors of the visual field test, as they are recorded by the video recorder. Moreover, some of the factors recorded by the operator may be analysed differently by another person afterwards, if necessary.

Preferably, the video recorder comprises one or more cameras with a viewing field of at least 8 cm, preferably of at least 10 cm and more preferably of at least 12 cm.

The size of the viewing field of the video recorder is chosen so as to allow the recording of the movements of at least part of patient's head, and in particular movements of both patient's eyes. It is then easier to analyse the response of the patient during the test. Moreover, in cases in which the dual task of fixating a central point and simultaneously detecting a stimulus presented in the periphery is not feasible by the patient, for instance a child, the large viewing field of the camera allows the change of the eye gaze direction to be recorded. In this case, the change of the eye gaze direction is used as a response of the patient when the stimulus is first presented in the centre of the screen and then in the periphery of the screen. In particular, such a test is not feasible on visual field systems currently available in clinics, because of the narrowness of the viewing field of their camera, which does not allow for head movement.

Preferably, the display for presenting visual stimuli is curved or flat.

According to a first embodiment, the camera is placed on one side of the display.

According to another embodiment, the display comprises a hole and the camera is placed behind said hole.

The camera, the position of the camera and the size of the viewing field of the camera are chosen so as to allow the recording of at least part of patient's head, and in particular of both patient's eyes and of their movements during the tests, without impairing performance of the test.

Preferably, the means for selecting the features of each stimulus determine the position, the luminosity, the colour or the size of the stimulus. Different parameters may be chosen for the successive stimuli, in order to check several perception skills of the patient. On one hand, the means for selecting the features of each stimulus may allow to make the test more sensitive: for instance, by reducing the luminance or size of the stimulus. On the other, in case of low vision, the means for selecting the features of each stimulus may increase the luminance or the size of the stimulus in order to make the test relevant. Some or all of the features of the successive stimuli may be recorded together with the time-codes of the video recorder.

Preferably, the system for testing the visual field of a patient also comprises a checking display for displaying the video of movements of at least part of the patient's head, and in particular movements of patient's eyes recorded by the video recorder. The checking display allows the visualisation and the analysis afterwards of the movements of the patient during the visual field test.

Preferably, the checking display is also for displaying, at the time-code recorded by the data recorder, the features of the stimulus viewed by the patient. In this case, the position of the stimulus and the response of the patient are both visualized on the checking display: such an embodiment eases the analysis of the visual field test, and especially the response time of the patient to each stimulus.

The checking display may also include five different representations of the visual field result: a first representation referred to as visual field representation, and a second representation referred to as camera representation. In the first representation, the positions of the stimuli are displayed on the checking display as they are seen from the patient's point of view. In the second representation, the positions of the stimuli are displayed on the checking display as they are seen from the point of view of the camera.

Preferably, in the first representation referred to as visual field representation, the checking display is for displaying the video of movements of at least part of the patient's head, and in particular movements of patient's eyes, and for overlaying the features of the stimuli on the video of the movements of at least part of the patient's head, and in particular movements of patients eyes. The aim of such a feature is to ease the interpretation of the visual field test afterwards thanks to the video of the movements of at least part of the patient's head, and in particular movements of the patient's eyes.

Alternatively, in the first representation referred to as visual field representation, the checking display may also be for displaying the video of movements of at least part of the patient's head, and in particular movements of patient's eyes, and for displaying the features of the stimuli next to the video of movements of at least part of the patient's head, and in particular movements of patient's eyes.

Preferably, in the second representation referred to as camera representation, the checking display is for horizontally flipping the position of the stimuli before overlaying the horizontally flipped position of the stimuli on the video of the movements of at least part of the patient's head, and in particular movements of patient's eyes, so as to make the horizontally flipped position of the stimuli coincident with the movements of at least part of the patient's head, and in particular movements of the patient's eyes displayed by the checking display. In this case, some or all of the features of each stimulus are displayed, by the checking display, in the expected direction of the patient's response movement recorded during the visual field test.

For instance, if the patient looks at the left bottom corner of the viewing field of his or her visual field, this will correspond to the left bottom corner in the visual field representation, and to the right bottom corner in the camera representation. Thus, in the camera representation, the features of the stimulus will be displayed at the right bottom corner, in the expected direction of the patient's response movement on the checking display.

Alternatively, in the second representation referred to as camera representation, the checking display may also be for horizontally flipping the position of the stimuli before displaying the features of the stimuli next to the video of movement of at least part of the patient's head, and in particular movements of patient's eyes.

Preferably, the data recorder is also for recording, for each visual stimulus, the response of the patient to the visual stimulus.

The response of the patient to the visual stimulus may be recorded by several methods: firstly by the patient himself, for instance by pushing a button when the stimulus is seen by him; secondly by the operator during the visual field test; and finally afterwards, by the person looking at the video. The recording of these different responses of the patient may ease the analysis of the video, for instance if the patient succeeded to fixate a central point and simultaneously detect a stimulus presented in the periphery.

In another aspect, the invention also provides a method for testing visual field of a patient, comprising:
a) selecting the features of visual stimuli,
b) presenting visual stimuli to the patient;
c) recording video of movements of at least part of patient's head, and in particular movements of patient's eyes, together with time codes, during the displaying of the visual stimuli, and
d) recording, for each visual stimulus, the features and at least one time-code of the video of the movements of at least part of the patient's head, and in particular movements of the patient's eyes, corresponding to the displayed visual stimulus.

Preferably, the method also comprises:
e) displaying the video of movements of at least part of the patient's head, and in particular movements of the patient's eyes together with the features of the stimulus viewed by the patient, at the time-code of the video of the movements of at least part of the patient's head, and in particular movements of the patient's eyes corresponding to the displayed visual stimulus.

Preferably, step e) comprises displaying the features of the stimuli next to the video of movements of at least part of the patient's head, and in particular movements of the patient's eyes, or overlaying the features of the stimuli on the video of the movements of at least part of the patient's head, and in particular movements of the patient's eyes.

Preferably, in step e), the positions of the stimuli are overlaid to the video of the movements of at least part of the patient's head, and in particular movements of the patient's eyes, and step e) also comprises horizontally flipping the position of the stimuli so as to make it coincident with the movements of at least part of the patient's head, and in particular movements of the patient's eyes.

Preferably, the method comprises:
f) looking at the video and at the features displayed in step e), and determining, for at least one visual stimulus, a response of the patient.

Preferably, the method comprises:
g) recording, for at least one visual stimulus, the response of the patent determined in step f).

### BRIEF DESCRIPTION OF THE DRAWINGS

The present invention and its advantages can be better understood on reading the following detailed description of a particular embodiment given by way of non-limiting example and illustrated by the accompanying drawings, in which:

FIG. 1 shows a schematic visual field system according to the invention;

FIG. 2 is an exempt of a preferred implementation for the viewing field of the camera;

FIG. 3 is an example of a presentation of a stimulus in the low right quadrant of the visual field of the subject and the corresponding eye movement response;

FIG. 4 is another example of a presentation of a stimulus in the low left quadrant of the visual field of the subject and the corresponding eye movement response; and

FIG. 5 is a schematic flow chart diagram illustrating one embodiment of a method according to the present invention.

### DETAILED DESCRIPTION OF THE INVENTION

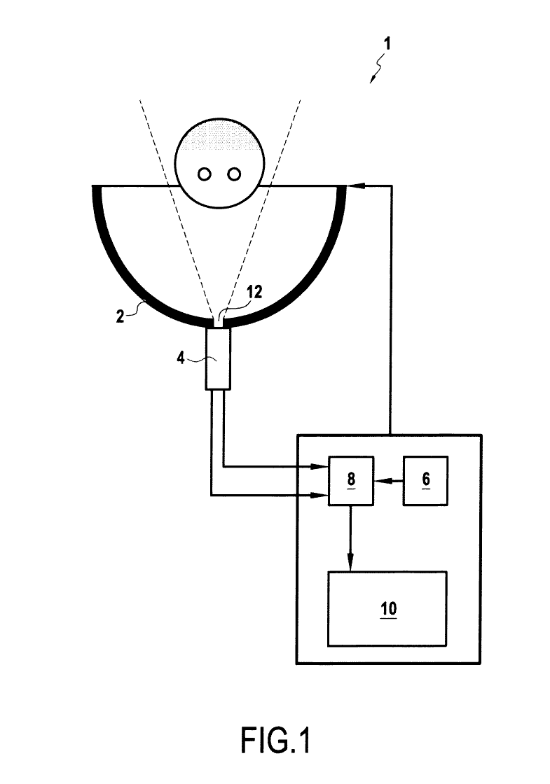

FIG. 1 shows an embodiment of a system 1 for testing visual field of a patient. The system 1 comprises a screen 2 for presenting a visual stimulus, for instance a circular spot of light, and a video recorder 4, for instance a camera, for recording movements of at least part of the patient's head, and in particular movements of the patient's eyes when the stimulus is presented on the screen 2, in the periphery of a patient's visual field. The system 1 also includes means 6 for selecting features of the stimuli presented on the screen 2, a data recorder 8 for recording data sent by the video recorder 4, and a checking display 10 for displaying the video of movements of at least part of the patient's head, and in particular movements of the patient's eyes.

The screen 2 is preferably a curved screen, for instance a hemispherical dome, on which successive visual stimuli are projected. The hemispherical dome allows eccentricities of 90°, which is approximately the limit of the physiological visual field.

Alternatively, the screen 2 may also be a flat screen, a LCD screen or OLED screen. However, with a flat screen, the tested visual field is limited as it can only reach eccentricities of about 20° to 30°.

The video recorder 4 may be a camera, and may be placed behind or near the screen 2. For instance, the screen 4 may comprise a hole 12 of small dimensions and allowing the video recorder 4 placed behind to collect the images of the patient through this hole 12. The hole 12 may have a diameter comprised between 1 mm and 4 cm, preferably between 5 mm and 2 cm. In case the hole 12 is positioned in the centre of the screen 4, the video recorder may provide a video focused on the patient's head.

Alternatively, the video recorder 4 may be placed above the screen 2, for instance above a flat screen 2.

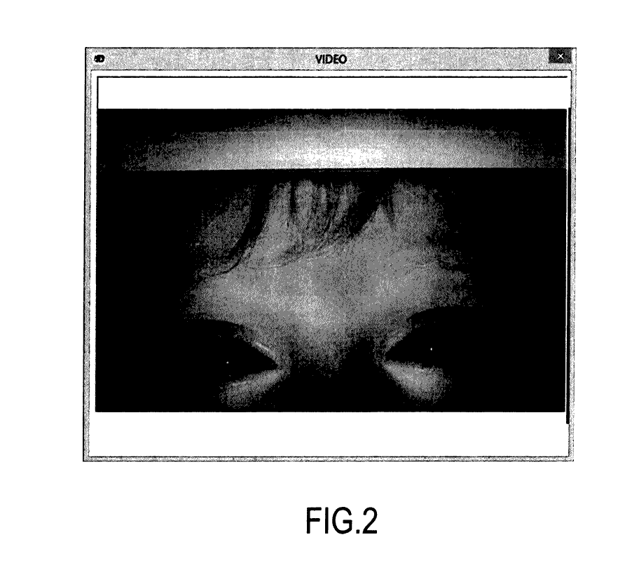

The camera of the video recorder 4 is chosen so as to have a field of view of at least 8 cm, preferably of at least 10 cm, and preferably of at least 12 cm. The aim of such a large visual field is to allow a binocular test, during which the eye movements of the patient are recorded by the video recorder 4. The binocular test eases the performing of the visual field test on uncooperative patients for instance a child, for which it is very difficult to impose immobility of the head, it thus allows better evaluation of movements of the patient during performance of the visual field test. Examples of the viewing field of the video recorder 4 are given at FIGS. 2 to 4: these figures show part of the head and both eyes of the patient during the test. It is then possible to analyse the reactions of the patient to the successive stimuli.

As mentioned above, during the visual field test, the video recorder 4 records movements of at least part of the patient's head, and in particular movements of the patient's eyes, but also time codes: time codes are a sequence of numeric codes generated at regular intervals by a timing synchronization system. Time codes allow to precisely determine the moment at which each picture of the video has been recorded, thus to synchronize the pictures of the video with the displaying of the successive stimuli on the screen 2. The video and the time codes recorded by the video recorder 4 are sent to the data recorder 8.

The features of the successive stimuli are selected by means 6. Means 6 may be a dedicated circuit or a processing unit that includes a programmable microprocessor. Means 6 may also be implemented in software performed by a computer microprocessor. Means 6 may list the features of visual stimulus for selection by the user, and send the selected features to the screen 2 for displaying by the screen 2. These features are, for instance, the position, size, color, luminance, duration, etc of each stimulus, and are sent by means 6 to the screen 2 for displaying. The displaying of visual stimuli may be controlled with a computer mouse or stylus interface allowing the operator to select the features of each stimulus. For instance, the operator may choose the position of the stimulus with the cursor of the mouse or stylus, and ensures the displaying by pressing the left button of a computer mouse.

The data recorder 8 records the data collected from the video recorder 4 and from the means 6 for selecting features of visual stimuli. In particular, the data recorder 8 allows recording of: video images of the movements of at least part of the patient's head, and in particular movements of the patient's eyes, the time codes of the video images and the features of the stimuli selected by means 6.

Furthermore, the response of the patient, determined by the operator during the test, may also be recorded by the data recorder 8, preferably together with the features of the corresponding stimulus. This can be done by the operator, for example, with a mouse right-click, or, in case of a lack of response of the patient, with a combination of a mouse right-click and simultaneously of a keyboard shift key.

For cooperative patients, the system 1 may also comprise a button (not represented) which can be used by the patient to indicate that the visual stimulus has been seen. This result of the test, given by the patient himself, may also be recorded by the data recorder, together with the features of the corresponding stimulus.

Thus, for each visual stimulus, the data recorder 8 may record, for instance, as a data vector, the following information: features of the visual stimulus (position, color, size, duration, luminance), time code of the video recorder 4, response provided by the patient and response provided by the operator.

The system 1 also comprises a checking display 10 for displaying the video of movements of at least part of the patient's head, and in particular movements of the patient's eyes recorded by the video recorder 4, and the features of the stimulus selected by means 6 and viewed by the patient, at the time-code of the video. The response, or lack of response, indicated by the operator or by the patient himself, may also be displayed by the checking display 10.

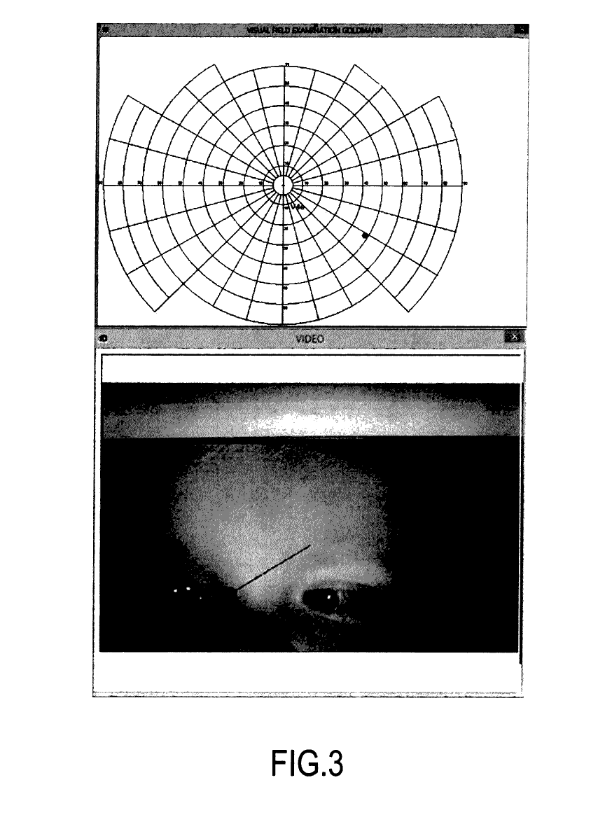

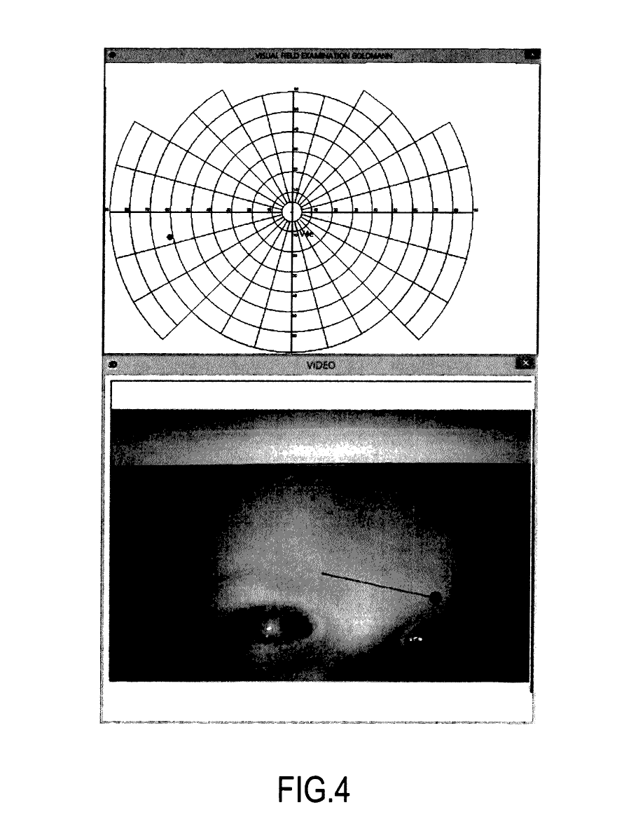

Preferably, both video and features of the stimuli are displayed at the same time, i.e. in synchrony, by the checking display 10. For instance, the checking display 10 may display a first window with the video of the test, and a second window with the features of the stimuli and the response of the patient. Examples of such windows are shown at FIGS. 3 and 4. In particular, at FIGS. 3 and 4, the top window displays the position of the stimulus in the visual field of the patient, which corresponds to what is seen by the patient during the test, and the bottom window displays the video recorded by the video recorder 4 at the same time. It is then possible to observe the reaction of the patient to the displaying of the visual stimulus.

In order to ease the analysis of the video, the checking display 10 may display the features of the stimuli next to the video, or may overlay the features of the stimuli on the video.

As can been seen at FIGS. 3 and 4, the top window displays the position of the stimulus in the visual field of the patient, but the bottom window also displays a vector representative of the position of the stimulus. In particular, in the checking display 10, the position of the stimuli is horizontally flipped so as to make it coincident with the movements of at least part of the patient's head, and in particular movements of the patient's eyes displayed by the checking display 10. Indeed, usually, the representation of the features of the stimuli corresponds to what is seen by the patient, whereas the video display of the eyes corresponds to what is recorded by the video recorder 4 placed in front of the subject. Therefore, when a stimulus is presented in the right side of the visual field of the patient, the movement of at least part of the patients head, and in particular movements of the patients eyes toward the stimulus corresponds to a movement of the image of the eye in the opposite direction (toward the left) on the checking display 18. In order to facilitate the interpretation of the patient's responses, the stimulus position is displayed as an overlay on top of the video of the eyes and is horizontally flipped so as to correspond to the eye movement response of the patient.

The checking display 10 displays the video of at least part of the patient's head, and in particular movements of the patient's eyes and the features of the stimuli in a synchronous manner.

Moreover, such a displaying also allows the operator to record other events.

The checking display may be used not only for a control of the test but also for an a posteriori interpretation. Indeed, during the replay of the test, the operator may add new response events or comments by using the same procedure as during the test. Alternatively, a snap shot of the video may be taken and included in an examination report by the operator. Such events, visible on the video and occurring during the test, may be for instance:
abnormal size of the pupil,
abnormal eye movements such as nystagmus,
presence of a ptosis,
abnormal position of the lens used to correct refractive errors
abnormal head position and head movements,
etc.

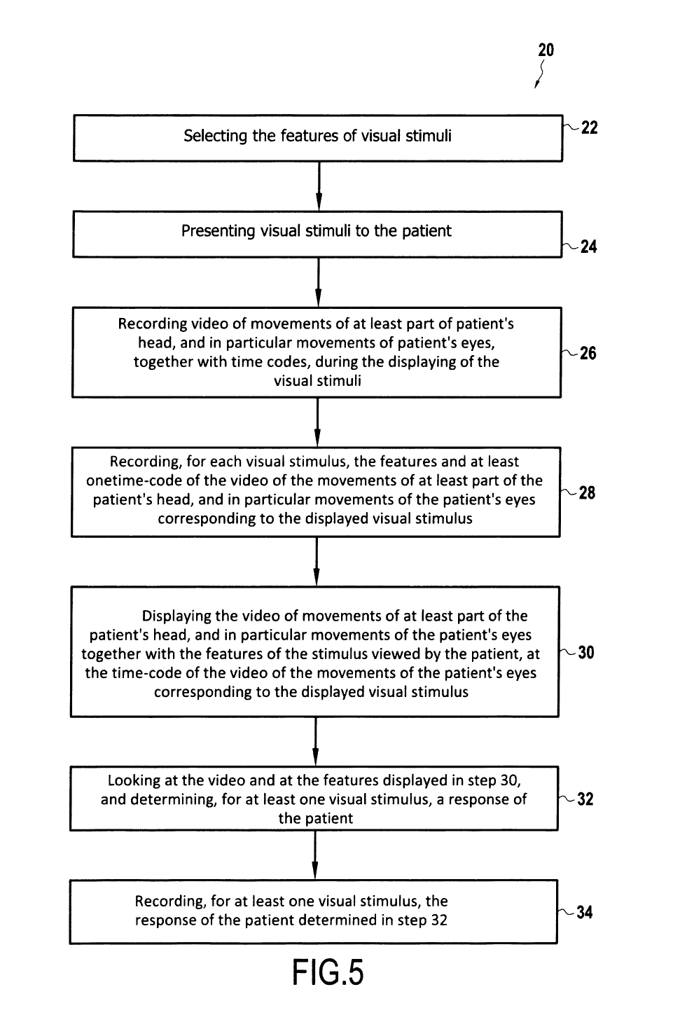

FIG. 5 is a schematic flow chart diagram illustrating one embodiment of a method 20 for testing visual field of a patient. In a first step 22 of the method 20, the features of visual stimuli are selected and then, in a second step 24, the visual stimuli are successively presented to the patient, at a given position on a screen. At the same time, in step 26, the video of at least part of the patient's head, and in particular of the movements of patient's eyes, are recorded, together with time-codes. Moreover, the response of the patient may also be recorded by the operator of the test, or by the patient himself if a button is provided to him for this purpose.

In step 28, for each visual stimulus, the features and at least one time-code of the moment corresponding to the displayed visual stimulus are recorded. For instance, the horizontal position, the vertical position, the size, the luminance, the color and the time code of the stimulus may be recorded. The response, or the lack of response, of the patient provided by the operator or by the patient himself may also be recorded together with the features of the stimulus.

In step 30, the video of movements of at least part of the patient's head, and in particular movements of the patient's eyes together with the features of the stimulus viewed by the patient at the time-code of the movements corresponding to the displayed visual stimulus, are displayed. In particular, in step 30, the features of the stimuli may be displayed next to the video of movements of at least part of the patient's head, and in particular movements of the patient's eyes, or may be overlaid on the video of the movements of at least part of the patient's head, and in particular movements of the patient's eyes. Preferentially, in step 30, the positions of the stimuli are flipped so as to make it coincident with the movements of at least part of the patient's head, and in particular movements of the patient's eyes and the positions of the stimuli are overlaid on the video of the movements of at least part of the patient's head, and in particular movements of the patient's eyes.

In step 32, the video and the features are looked at and, for at least one stimulus, a response of the patient is determined.

In step 34, the response of the patient determined in step 32 is recorded.

Thus, thanks to the system, and the corresponding method, it is possible to perform a visual field test on uncooperative patients, in particular on children, by recording eye movements of the patient. The test relies on the natural reaction of the patient, typically a child, to perform a gaze toward a visual stimulus that be detects in his peripheral visual field. With the video recording of the movements of at least part of the patient's head, and in particular movements of the patient's eyes, the person analysing the test is able to detect a response of the patient: the movement of the eye indicates whether the patient perceives the stimulus or not. It is then easier to perform the test and to analyse it, even if the patient is not able to learn specific tasks.

1. A system for testing visual field of a patient, comprising:
means for selecting the features of successive visual stimuli,
a display for presenting said successive visual stimuli to the patient;
a video recorder for recording video of movements of at least part of the patient's head together with time codes, during the presentation of said visual stimuli, and
a data recorder for recording, for each visual stimulus, the features and at least one time-code of the video recorder corresponding to the displayed visual stimulus.

2. A system according to claim 1, wherein the video recorder comprises one or more cameras with a viewing field of at least 8 cm.

3. A system according to claim 1, wherein the video recorder comprises one or more cameras with a viewing field of at least 10 cm.

4. A system according to claim 1, wherein the video recorder comprises one or more cameras with a viewing field of at least 12 cm.

5. A system according to claim 1, wherein the display for presenting visual stimuli is curved.

6. A system according to claim 1, wherein the display for presenting visual stimuli is flat.

7. A system according to claim 1, wherein a camera is placed on one side of the display.

8. A system according to claim 1, wherein the display comprises a hole and a camera is placed behind said hole.

9. A system according to claim 1, wherein the means for selecting the features of each stimulus determine the position, the luminosity, the colour or the size of the stimulus.

10. A system according to claim 1, further comprising: a checking display for displaying the video of movements of at least part of the patient's head recorded by the video recorder.

11. A system according to claim 10, wherein the checking display is also for displaying, at the time-code recorded by the data recorder, the features of the stimulus viewed by the patient.

12. A system according to claim 11, wherein the checking display is for displaying the video of movements of at least part of the patient's head, and overlaying the features of the stimuli on the video of the movements of at least part of the patient's head.

13. A system according to claim 12, wherein the checking display is for horizontally flipping the position of the stimuli before overlaying the horizontally flipped position of the stimuli on the video of the movements of at least part of the patient's head, so as to make the horizontally flipped position of the stimuli coincident with the movements of at least part of the patient's head displayed by the checking display.

14. A system according to claim 1, wherein the data recorder is also for recording, for each visual stimulus, the response of the patient to the visual stimulus.

15. A system according to claim 1, wherein the at least part of the patient's head is the patient's eyes.

16. A method for testing visual field of a patient, comprising:
a) selecting the features of visual stimuli,
b) presenting visual stimuli to the patient;
c) recording video of movements of at least part of patient's head together with time codes, during the displaying of the visual stimuli, and
d) recording, for each visual stimulus, the features and at least one time-code of the video of the movements of at least part of the patient's head corresponding to the displayed visual stimulus.

17. A method according to claim 16, comprising:
e) displaying the video of movements of at least part of the patient's head together with the features of the stimulus viewed by the patient, at the time-code of the video of the movements of the patient's eyes corresponding to the displayed visual stimulus.

18. A method according to claim 17, wherein step e) comprises displaying the features of the stimuli next to the video of movements of at least part of the patient's head.

19. A method according to claim 17, wherein step e) comprises overlaying the features of the stimuli on the video of the movements of at least part of the patient's head.

20. A method according to claim 19, wherein step e) also comprises horizontally flipping the position of the stimuli so as to make the position of the stimuli coincident with the movements of at least part of the patient's head.

21. A method according to claim 17, comprising:
f) looking at the video and at the features displayed in step e), and determining, for at least one visual stimulus, a response of the patient.

22. A method according to claim 21, comprising:
g) recording, for at least one visual stimulus, the response of the patient determined in step f).

23. A method according to claim 16, wherein the at least part of the patient's head is the patient's eyes.
